(19) United States
(12) Patent Application Publication
(10) Pub. No.: US 2018/0206851 A1
(43) Pub. Date: Jul. 26, 2018

(54) Hydrogel intrasaccular occlusion device

(71) Applicant: Daniel E. Walzman, Bergenfield, NJ (US)

(72) Inventor: Daniel E. Walzman, Bergenfield, NJ (US)

(21) Appl. No.: 15/932,580

(22) Filed: Mar. 15, 2018

Related U.S. Application Data
(63) Continuation-in-part of application No. 15/732,147, filed on Sep. 26, 2017.
(60) Provisional application No. 62/496,505, filed on Oct. 19, 2016.

(51) Int. Cl. A61B 17/12; A61B 17/00; A61L 31/14; A61L 31/06; A61L 31/02

(52) U.S. Cl. CPC A61B 17/12145; A61B 17/12031; A61B 17/12113; A61B 17/1215; A61B 17/0057; A61L 31/146; A61L 2430/36; A61L 31/06; A61L 31/022; A61B 2017/00893; A61B 2017/00942; A61B 2017/00592; A61L 31/145

(57) ABSTRACT

The present disclosure relates to the field of endovascular treatment. More particularly, the present invention discloses a tool designed to implement an endovascular treatment capable of delivering medicine, such as amorphous hydrogel to wounds proximate to injury caused by endovascular treatment. Said hydrogel is affixed to an endovascular device.

### CROSS-REFERENCES

This is a continuation-in-part application claiming priority to nonprovisional utility application Ser. No. 15/732,147 filed Sep. 26, 2017 (26 Sep. 2017), which claims priority to provisional application Ser. No. 62/496,505 filed Oct. 19, 2016 (19 Oct. 2016)

### FEDERALLY FUNDED R&D

None

### BACKGROUND OF THE INVENTION

### Field of the Invention

The present disclosure relates to the field of endovascular treatment. More particularly, the present invention is a tool designed to implement an endovascular treatment.

The present invention is a tool to safely and effectively implement an endovascular treatment. The prior art includes endovascular devices have provided high density, mesh-like metallic materials across the aneurysm neck, in place of coil technology. It has also taught in vivo preclinical performance of a self-expanding intrasaccular embolization devices (see Preliminary Results of the Luna Aneurysm Embolization System in a Rabbit Model: A New Intrasaccular Aneurysm Occlusion Device by S. C. Kwon in the American Journal of Neuroradiology AJNR 2011 32: 602-606). While the devices identified in the prior art achieved high rates of successful deployment, medical difficulties still arose due to clot formation in the normal vessel adjacent to the device, poor adhesion to the aneurysm walls with resultant endoleak, and/or continued filling of the aneurysm, and/or delayed occlusion of the aneurysm, and/or delayed compaction of the device, and/or delayed dislodging of the device—all of which present the risk of potentially fatal aneurysm rupture.

### Background

The present invention substantially fulfills the forgoing unmet needs. A gel is a solid jelly-like material that can have properties ranging from soft and weak to hard. A hydrogel is a network of polymer chains that are hydrophilic, sometimes found as a colloidal gel in which water is the dispersion medium. Hydrogel dressings consist of 90 percent water in a gel base, and serves to help monitor fluid exchange from within the wound surface.

The application of hydrogel assists in protecting areas adversely affected during endovascular treatments from wound infection and promotes efficient healing. Hydrogel dressings generally come in three different forms (which constitute various release mechanisms), including: amorphous hydrogel: a free-flowing gel, distributed in tubes, foil packets and spray bottles; impregnated hydrogel: typically saturated onto a gauze pad, nonwoven sponge ropes and/or strips; and sheet hydrogel: a combination of gel held together by a thin fiber mesh. These may be woven and/or adhered to metal structures as well.

In addition to aiding wound treatment, hydrogel has been shown to offer relief from pain for hours after application. Furthermore, the expansion of the hydrogel after it is implanted into the body may increase the coverage of a metal mesh implanted and thereby decrease permeability of blood into the aneurysm, promoting faster thrombosis and healing of the aneurysm.

### PRIOR ART

The prior art discloses the use of hollow structures to ameliorate the difficulties associated with vascular defects. More particularly, the prior art discloses the coating of intravascular structures with hydrogel to facilitate the use of structures to ameliorate said difficulties. Such coating is a known technique that unfortunately is subject to collapse of the structure. No prior art teaches the use of hollow structures utilizing hydrogel to prevent collapse, rather the prior art teaches the use of hollow structures utilizing hydrogel and blood contained within the hollow structure to prevent collapse.

For example, Phillipe Marchand et al. (US2009/0275974 and US20110152993A1) teach a hollow medical device which is subject to collapse. Conversely, the present invention expressly teaches the use of a semipermeable coating of the medical device with hydrogel to allow the option of a hollow or filled medical device to prevent its collapse and allows the infusion of additional packing material and/or medicine.

Marchand teaches the use of a biocompatible metal and polyethylene terephthalate (PET or PETE) for the purpose of forming a device to withstand collapse when used to close aneurysms, whereas the present invention uses those materials for the purpose of forming a semipermeable coating to allow the exclusion of blood and the insertion of additional support material and medicine into the medical device to prevent collapse.

The prior art also teaches a variety of other uses of hydrogel to ameliorate vascular defects. For example, see Brian J. Cox (U.S. Pat. Publ. no. 2015/0142042) (an Aneurysm treatment device and method of use) and Gregory Cruise et al. (U.S. Pat. Publ. no. 2005/0196426) (Hydrogels that undergo volumetric expansion in response to changes in their environment and their methods of manufacture and use). However, such treatments are suboptimal because hydrogel undergoes biometric expansion in response to its environment, the methods of manufacturing and use. A need exists for a control mechanism which will allow the addition or subtraction of hydrogel to overcome said difficulty. A need exists for a medical device applying hydrogel to the medical device to prevent collapse.

Burke et al. (U.S. Pat. Publ. no. 2005/0033409) teaches an aneurysm treatment device and method of use using a hollow medical structure with varying of thicknesses of hydrogel. A need exists for a medical device applying hydrogel through lumens or a medical device composed of hydrogel with a non-hydrogel initial shaping element.

The present invention teaches the use of a semipermeable membrane created using hydrogel or other materials. Said semipermeable membrane may be a unidirectional membrane allowing medicine and packing material from the hollow formed by the mesh element of the present invention to flow beyond the surface of said mesh into the void between the mesh and the walls of the vascular defect. Said membrane would not allow blood to flow into said void, which void could be filled with hydrogel or other packing material.

The permeability of the mesh may be increased by adding at least one hydrogel layer to the interior surface of the intrasaccular device. Said layer by be affixed to the interior surface prior to insertion of the device or may be affixed using material introduced after the insertion through the lumens in wire (12).

The expansion of the hydrogel can also improve adhesion to the aneurysm wall, reducing the risk of endoleaks and aneurysm filling and/or rupture, while also reducing the risk of the device being dislodged further into the aneurysm or elsewhere. The expansion of the hydrogel both along the perimeter of the mesh as well as internally will also promote faster complete thrombosis and occlusion of the aneurysm, reducing the risk of subsequent aneurysm rupture. Additionally, hydrogel is less thrombogenic than metal, so a thin layer on any surface exposed to blood flow external to the aneurysm can also reduce the risk of thromboembolic complications that can otherwise be caused by platelet and other clotting factors adhering to metal in the body.

Additionally, the expansion of the hydrogel increases the rigidity of the device after deployment, allowing less risk of the device collapsing, dislodging, and/or compacting, while not increasing the rigidity before deployment, which may increase the difficulty and risk of deploying said device. The present invention expressly teaches the filling of the medical device with hydrogel to prevent the collapse of the medical device.

Still further, similar advantages would exist when using said coating of hydrogel on a Left atrial appendage (LAA) closure device, such as a Watchman (Boston Scientific) or similar device. This would improve the success rate, since hydrogel can expand to fill the LAA, so less precise deployments could be acceptable. It also would result in more immediate reduction in clots forming. Currently, 45 days of Coumadin is recommended after implantation, followed by 6 months of Plavix. The need for such continued anticoagulation and strong anti-platelets, with their associated risks of bleeding complications, can be reduced by the addition of a coating of hydrogel, which is less thrombogenic than metals, plastic, polyesters, polyethylene terephthalate (PETE or Dacron), and other materials commonly used in medical devices.

A study published in the Journal of the American College of Cardiology: Basic to Translational Science, reported that an inject-able gel can maintain its healing characteristics. In particular, rebuilding of muscular structures was reported from a gel originally derived from a pig's cardiac muscle tissue, which was stripped of cells until all that was left was an extracellular matrix. A 2010 study in the Journal of Cell Science noted that an element of gel used in the aforementioned Journal of the American College of Cardiology study was responsible for tissue regeneration and re-growth. One non-limiting version of a hydrogel that expands in the body is a co-polymer of acrylamide and sodium acrylate cross linked.

A major difference in this device is the affixation of hydrogel to the device. In the prior art hydrogel is merely applied. In the current invention, hydrogel may be affixed on the inside and/or outside of any stent. Hydrogel treatment may be useful only on the inside or the outside of the stent depending upon the relative size of the vessel and the device.

Outside affixation prevents or ameliorates adverse interaction between the outer surface of the stent and tissue with which it may come in contact, thereby inflaming and thickening the tissue. Inside affixation helps decrease thrombus formation and in-stent stenosis.

Stents and other endovascular devices have issues in that they are thrombogenic when they are first inserted, until they are incorporated into the vessel/endothelialized, or in some cases such as mechanical cardiac valves, forever. This results in significant rates of thrombotic complications, including thrombosed vessels resulting in stroke, myocardial infarction, or other ischemic complications. In order to minimize such risks patients are routinely started on anti-platelet therapy, often dual anti-platelet therapy with agents such as Plavix or Brilinta, and aspirin. In addition, other endovascular devices, particularly those implanted in the heart such as mechanical heart valves, tend to cause a different type of clot that necessitates the use of anticoagulants to protect against clot formation. Although the medications reduce the rate of clot formation, they do not eliminate clot formation altogether and patients can still suffer complications from clotting. Additionally, all these medications have significant rates of bleeding complications. Hydrogel is more inert and does not cause thrombus formation/induction.

Thus, the current invention discloses placement of a thin coating of hydrogel on the entire surface of any endovascular device exposed to the inner surface of the blood vessel and/or blood products. In other embodiments, this would include a layer of hydrogel over a portion of such a device as well. The latter may reduce but not completely eliminate the risk of thrombus formation. By completely covering these devices with the thin layer of hydrogel, the device may significantly reduce the rate of thrombus formation and thus reduce the need for anti-platelet and or anticoagulant. Anti-platelet and anticoagulant medications have significant associated morbidity. By eliminating the need for these elements, a reduction in morbidity may be achieved.

Other embodiments of endovascular devices of the current invention include a layer of hydrogel affixed to other surfaces including inner and outer surfaces of metal stents, as well as covered stents, cardiac valves, left atrial appendage occlusion devices such as the Watchman, intra-saccular aneurysm devices, pressure monitors, wires/Leeds Etc. Covering metals, and/or plastics, and/or polyesters, and/or Dacron, and combinations thereof.

If the hydrogel is placed around all surfaces, including the surface pressing on the vessel wall, it may reduce the rate of intimal hyperplasia caused by the vessel reacting to the foreign body. Intimal hyperplasia causes vessel narrowing and/or occlusions, in some cases only on outer walls.

In sum, the hydrogel may be placed on the exterior of devices for use invasive medical procedures. Hydrogel may also be affixed to the interior of medical devices for use in such procedures. Hydrogel may be affixed to both the interior and exterior of medical devices for use in appropriate procedures.

The thickness of the affixed hydrogel layer may differ between the interior and exterior. The unhydrated, pre-insertion thickness of the hydrogel is typically from approximately one nanometer to approximately one centimeter. Thickness need not be uniform along the surface.

The current invention may be used in heart valves and other devices housed within bodies.

The present invention envisions adding a hydrogel to a mesh-like saccular aneurysm embolization device, such as the Sequent Web, the Luna Aneurysm Embolization system or similar devices or systems. Once done, and deployed in the body the hydrogel expands and further decreases the permeability of the device to blood. The permeability is a function of the amount of hydrogel and the thickness of the hydrogel on the mesh. Said amount and thickness may be increased or decreased using the lumens inside wire (20). This can facilitate more immediate thrombosis of the aneurysm, resulting in more immediate reduction in the risk of the aneurysm rupturing. It may also help facilitate subsequent vessel healing over the device, and thereby may reduce the risk of recurrence.

In the prior art, such as Marchand et al. (US20110152993A1), a globular device conforms to adapt to irregular shaped vascular defects such that about three-quarters of more of the vascular defect volume is occluded by a combination of device and blood contained therein. The current invention uses one or more layers of hydrogel or other materials to create various levels of shell permeability. In one embodiment, the current invention achieves impermeability of the hollow interior of the mesh using hydrogel.

The hollow space can be filled with medication to promote shrinkage of a vascular defect such as an outpouching in the body, aneurysm, or other, including in the left atrial appendage, or diverticulae of other organs. The hollow space can also be filled with packing material that allows structural support. Said packing material may be subsequently removed and replaced with medication.

Marchand limits the filling to blood. The present invention allows other options such as packing material and medication. Marchand has no element to remove blood filling the device, the present invention teaches filling and removing system using the lumens running therethrough.

With respect to the prior art, in particular Marchand '974 teaches the use of hydrogel for the purpose of reducing the porosity of a medical device. Specifically, Marchand teaches the application of hydrogel to the shell (40) of a hollow medical device to clog the holes in and around said medical device while keeping the interior of said medical device open. More specifically, Marchand teaches a particular range of hydrogel thickness to prevent the clogging of the interior of the medical device. The present invention expressly teaches the filling of the medical device with hydrogel to prevent the collapse of the medical device, whereas Marchand teaches away from using hydrogel to do the same.

The present invention differs from Marchand in other ways as well. For example, Marchand teaches the use of hydrogel to create various levels of shell (40) permeability for the exterior surface of the medical device taught by the invention, while the present invention teaches the use of hydrogel to make the shell of the medical device to sometimes be completely impermeable.

Additionally, Marchand allows: “Active materials such as a responsive hydrogel may be attached or otherwise incorporated into permeable shell 40 of some embodiments such that it swells upon contact with liquids over time to reduce the porosity of the permeable shell 40”, whereas the present invention teaches the use of hydrogel to both fill holes in the device and fill the medical device completely which will prevent potential collapse of said shell which has occurred in clinical practice.

While Marchland teaches the use of hydrogel simply to close pores, the present invention teaches the use of hydrogel to expand between the outer walls of the shell and the walls of the aneurysm and any associated radial out-pouching. This teaching has the potential for statistically significant improvements in treatment outcomes by preventing endoleaks around the shell (which would result in continued or recurrent aneurysm opacification (filling), with the associated risk of rupture, as taught by U.S. Pat. No. 9,775,730 to Walzman, wherein claim 10 recites “ . . . wherein the covering flow-impeding material comprises a hydrogel on its outer surface that is adapted for expansion to fill any empty spaces between the covering flow-impeding material and the vessel wall for minimization of a risk of an endoleak after deployment of the endovascular device.” In addition, claim 17 states “ . . . wherein the covering flow-impeding material comprises a hydrogel on its outer surface that is adapted for expansion to fill any empty spaces between the covering flow-impeding material and the vessel wall for minimization of a risk of an endoleak after deployment of the endovascular device.”

The present invention teaches the use of adding additional packing material via lumens in wire (12) with sufficient pressure in the wire (12) lumens, packing material will be pushed from the interior of the device through the mesh walls to fill the space between the device and the walls of the outpouching or other vascular defect. In some embodiments, sufficient amounts of hydrogel may be affixed to the inner and outer surfaces of the mesh to allow said hydrogel to expand beyond the mesh wall to fill the space between the device and the walls of the outpouching or other vascular defect.

The present invention teaches the use of hydrogel to expand from the top of the shell of a medical device to fill the upper portions of the aneurysm and any associated outpouching, which will further aid in durable aneurysm closure, and will also further prevent potential dislodgement of said shell further into said aneurysm, which can result in aneurysm recurrence and subsequent injury to the patient. Marchand teaches the use of hydrogel for the purpose of limiting (but not closing) pores in the exterior shell of a medical device, nor does Marchand teach the use of hydrogel to close aneurysms.

Marchand teaches the use of hydrogel for the purpose of limiting pore size in the exterior shell of a medical device. Marchand does not teach the use of hydrogel to secure the medical device in a desired position, nor does it teach the coating of exposed surfaces of said medical device. The present invention teaches both. The present invention expressly teaches the application of a thin layer of hydrogel on a medical device for the purpose of having said hydrogel expand between the device and the tissue wall, to prevent endoleaks and better secure said device in the desired position. A thin layer of hydrogel coating also prevents the blood from exposure to the metal or other device material thereby reducing the thrombogenicity of said device, and reducing the need for antiplatelet and/or anticoagulant medications (see spec. para. 8).

Marchand does not teach the use of hydrogel as a medium to carry medications. The present invention teaches the use of hydrogel as a medium to carry medication which can slowly leach into the body over a given time. The present invention also teaches that the coating may include hydrogel containing vasodilators such as Verapamil that would be slowly released over two to three weeks that can be implanted in carotid arteries and/or vertebral arteries after a ruptured brain aneurysm, to reduce the incidence of symptomatic intracranial vasospasm.

### BRIEF DESCRIPTION OF THE DRAWINGS

The invention will be better understood and objects other than those set forth above will become apparent when consideration is given to the following detail description thereof. Such description makes reference to the annexed drawings wherein:

FIG. 1 showing at distal end of wire (12) deployed device (10) designed to implement an endovascular treatment mesh (20) at treatment site with gel coat (22); optional lumens in wire (12) are not shown.

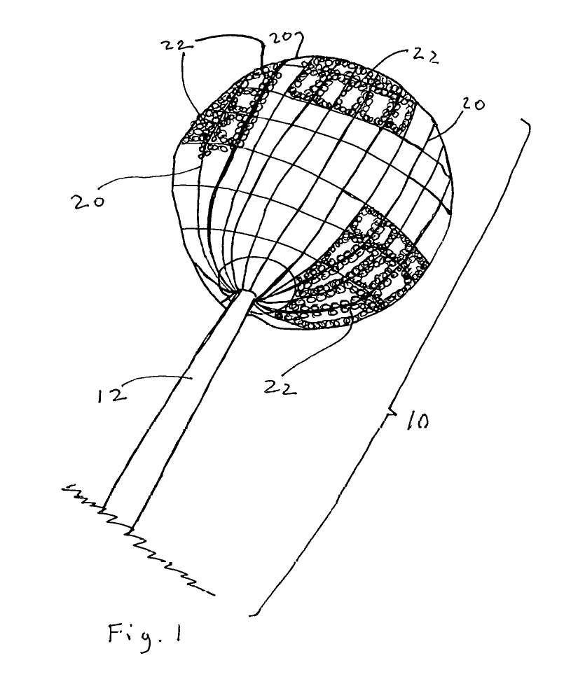

FIG. 2 showing at distal end of wire (12) showing undeployed device designed to implement an endovascular treatment mesh (20) with gel coat (22).

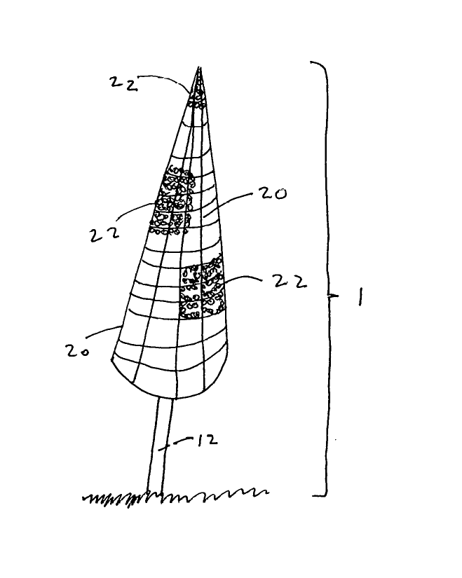

### DETAILED DESCRIPTION OF THE INVENTION

The present disclosure teaches the placement of amorphous hydrogel within or coating surfaces of a device designed to implement an endovascular treatment. Said amorphous hydrogel is adhered to select surfaces of said device designed to implement an endovascular treatment and/or is contained by said device designed to implement an endovascular treatment, or both. When said coated designed to implement an endovascular treatment is proximately positioned at the treatment point, and the metal mesh device such as the Sequent Web or Luna Aneurysm Embolization system or similar system is deployed in the body, the exposure of the adhered added hydrogel with the device to the blood and temperature in the body causes it to expand further, decreasing the permeability of the device to blood and promoting more immediate thrombosis of the aneurysm, which results in more immediate decrease in the risk of the aneurysm rupturing.

Wire (12) may be solid or channeled with lumens. Said lumens are capable of delivering medication and packing materials. Said lumens are also capable of evacuating said packing materials and other material inside the device's hollow volume.

It will be understood that the above particular embodiment is shown and described by way of illustration only. The principles and the features of the present disclosure may be employed in various and numerous embodiments thereof without departing from the scope and spirit of the disclosure as claimed. The above-described embodiment illustrated the scope of the disclosure but does not restrict the scope of the disclosure.

What is claimed is:

1. A device having an expandable mesh shell deployed upon a wire, said shell covered in at least one material adapted to create a semipermeable hollow sphere adapted to close a vascular defect.

2. The device of claim 1, wherein said at least one material is hydrogel.

3. The device of claim 1, wire has at least one lumen communicating the ends of said wire.

4. The device of claim 3, wherein said lumens are adapted to deliver material therethrough.

5. The device of claim 2, wherein at least one layer of said hydrogel is affixed to an exterior surface of said intrasaccular device.

6. The device of claim 2, wherein at least one layer of said hydrogel is affixed to an interior surface of said intrasaccular device.

7. The device of claim 2, wherein at least one layer of said hydrogel is affixed to an interior surface and an exterior surface of said intrasaccular device.

8. The device of claim 2, wherein the thickness of at least one layer of said hydrogel decreases from the proximal to the distal end of said device.

9. The device of claim 2, wherein the thickness of at least one layer of said hydrogel decreases from the distal end to the proximal end of said device.

10. The device of claim 3, wherein the thickness of at least one layer of said hydrogel increases between the distal end to the proximal end of said device.

11. The device of claim 2, wherein the thickness of at least one layer of said hydrogel decreases between the distal end to the proximal end of said device.

12. The device of claim 2, wherein the thickness of at least one layer of said hydrogel is uniform from the distal end to the proximal end of said device.

13. The device of claim 2, wherein said hydrogel further includes at least on vasodilator.

14. The device of claim 1, adapted to treat intracranial saccular aneurysms.

15. The device of claim 1, adapted to treat extracranial saccular aneurysms.

16. The device of claim 1, adapted to treat arterial or venous aneurysms.

17. The device of claim 1, adapted for use in left atrial appendage closure.

18. The device of claim 1, adapted for use in an outpouching in the body.

19. The device of claim 1, adapted for use in diverticulae of other organs.

20. The device of claim 1, wherein said at least one material includes a biocompatible metal.

21. The device of claim 1, wherein said at least one material includes a biocompatible polyester.

22. The device of claim 17, wherein said polyester comprises polyethylene terephthalate (PETE or Dacron).

23. The device of claim 1, wherein said at least one material comprises a combination of a biocompatible metal and a biocompatible polyester.
